(19) United States
(12) Patent Application Publication
(10) Pub. No.: US 2008/0306057 A1
(43) Pub. Date: Dec. 11, 2008

(54) P13K Inhibitors for the Treatment of Endometriosis

(75) Inventors: Stephen Palmer, Plympton, MA (US); Selvaraj Nataraja, Randolph, MA (US)

Correspondence Address: BAKER & DANIELS LLP, 805 15TH STREET, NW, SUITE 700, WASHINGTON, DC, 20005, US

(73) Assignee: LABORATORIES SERONO SA, Aubonne (CH)

(21) Appl. No.: 12/066,043

(22) PCT Filed: Aug. 28, 2006
(86) PCT No.: PCT/US06/33679

§ 371 Date: Jul. 10, 2008

(30) Foreign Application Priority Data
Oct. 11, 2005 (EP) 05109448.0

(51) Int. Cl. A61K 31/427; A61K 31/426; A61K 31/538; A61K 31/517; A61K 31/498; A61K 31/4439; A61P 43/00

(52) U.S. Cl. 514/230.5; 514/369; 514/266.24; 514/249; 514/314

(57) ABSTRACT

This invention relates to a method of treating and/or preventing endometriosis comprising administering a PI3K inhibitor. The PI3K inhibitor can also be administered combined with a hormonal suppressor. The invention further relates to the treatment of endometriosis-related infertility.

### BACKGROUND OF THE INVENTION

Endometriosis is one of the most frequent diseases of women in their reproductive lifespan. It is characterized by the presence of endometrial tissue outside the uterine cavity, consisting histological of glands and stroma. The anatomical sites most often affected are the ovaries, uterosacral ligaments, pelvic peritoneum, rectovaginal septum, cervix, vagina, the fallopian tubes and vulva.

Endometriosis is considered to be a benign disease, but endometriotic lesions occasionally become malignant. As in other kind of malignancies, the development of endometriosis-derived neoplasms is due to concurrent events, involving alterations in growth factors and/or oncogenes regulation (Kyama e al. 2003). Further, endometriosis is considered as a major cause of infertility (Giudice et al. 2004).

The current treatment of endometriosis consists of hormonal therapy and/or surgery. Hormonal therapies include high dose of progestogens, oral contraceptives (combinations of estrogen and progesterone), Danazol (an androgenic derivative of ethisterone) and more recently GnRH agonists. These hormonal therapies are effective on pelvic pain and may induce an objective regression of lesions, but have several caveats. Estrogen may stimulate and cause proliferation of endometriotic tissue since it may be unable to respond to progesterone (Dawood et al, 1993). Progestational agents can provoke irregular bleeding along with depression, weight gain, and fluid retention. Danazol can improve symptoms in approximately 66-100% of the patients suffering from pain, but recurrence rates after up to 4 years are approximately 40%-50%. Other drawbacks of Danazol therapy are weight gain and androgenic side effects. GnRH analogs are more potent and long acting than native GnRH, which act by removing the estrogenic stimulus for the growth of all estrogen sensitive tissues. Side effects of GnRH analogs are mainly secondary to the profound hypoestrogenemia, like decreased bone density, and recurrence rate are up to 50% after 5 years (Waller et al., 1993).

Surgical intervention can be conservative, if fertility is desired, or can lead to the removal of the uterus, tubes and ovaries in case of severe disease. In any case, even limited surgical treatment leads to a significant decrease in fertility.

Although endometriosis stands as one of the most investigated disorders of gynecology, the current understanding of pathophysiology of the disease remains elusive. According to a favored theory, endometriotic lesions develop by eutopic endometrical cells leaving their primary site, possibly by retrograde menstruation, and implant at distant sites, followed by invasion of host tissue and proliferation. Furthermore, it appears that endometriosis is an invasive and metastasizing disease. Though endometriotic cells proliferate to a certain extent, they are not neoplastic as typically found in carcinomas. Apparently, endometriotic cells become senescent, apoptotic and necrotic. Inflammatory responses that are induced or accompanied by lesion formation finally lead to fibrosis and the formation of scars.

Survival of the ectopic implants is due to a reduced cell death (apoptosis) of these implants, and is presumed to be due to increased expression of survival cell signaling pathways. Proteins or specific small molecule compounds that induce target-specific cell-death of ectopic endometriotic cells without affecting eutopic endometrium or other normal cells could be used as a treatment for eliminating endometriosis. In this regard, the effect of PI3K inhibitors on their ability to induce cell death of endometriotic cells, an immortalized human epithelial endometriotic cell, was examined.

PI3Ks (Phosphoinositide 3-kinases) have a critical signalling role in cell proliferation, cell survival, vascularization, membrane trafficking, glucose transport, neurite outgrowth, membrane ruffling, superoxide production, actin reorganization and chemotaxis (Cantley (2000) and Vanhaesebroeck (2001)). PI3K consists of two subunits, a catalytic P110 subunit and a regulatory and localizing subunit, P85. The major catalytic function of the PI3K is in the P110 subunit that acts to phosphorylate inositol phospholipids (PIP2: phosphatidyl inositol 4,5 bis-phosphate) in the plasma membrane at the 3 position within the inositol sugar ring. The inositol phospholipids (phosphoinositides) intracellular signalling pathway begins with binding of a signalling molecule (extracellular ligands, stimuli, receptor dimerization, transactivation by heterologous receptor (e.g. receptor tyrosine kinase) to a G-protein linked transmembrane receptor integrated into the plasma membrane. PI3K converts the membrane phospholipid PIP(4,5)₂ into PIP(3,4,5)₃ which in turn can be further converted into another 3′ phosphorylated form of phosphoinositides by 5′-specific phosphoinositide phosphatases, thus PI3K enzymatic activity results either directly or indirectly in the generation of two 3′-phosphoinositide subtypes that function as second messengers in intra-cellular signal transduction.

The evolutionary conserved isoforms P110α and β are ubiquitiously expressed, while δ and γ are more specifically expressed in the haematopoetic cell system, smooth muscle cells, myocytes and endothelial cells (Vanhaesebroeck 1997). Their expression might also be regulated in an inducible manner depending on the cellular-, tissue type and stimuli as well as disease context.

To date, eight mammalian PI3Ks have been identified, divided into three main classes (I, II, and III) on the basis of sequence homology, structure, binding partners, mode of activation, and substrate preference in vitro.

Two compounds, LY294002 and wortmannin are known PI3-kinase inhibitors. These compounds are non-selective PI3K inhibitors.

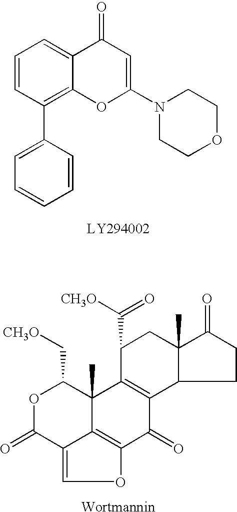

Azolidinone-vinyl benzene derivatives, which are described in WO 04/007491, and 2-imino-azolinone-vinyl fused-benzene derivatives, which are described in WO 05/011686 are said to be PI3 Kinase inhibitors, in particular of PI3 Kinase gamma. These compounds are said to be useful in the treatment and/or prophylaxis of autoimmune disorders and/or inflammatory diseases, cardiovascular diseases, neurodegenerative diseases, bacterial or viral infections, kidney diseases, platelet aggregation, cancer, graft rejection or lung injuries.

The invention described herein clearly shows the unexpected result that inhibiting PI3K, by means of a PI3K inhibitor, reduces endometriosis. The reduction of endometriotic lesions using PI3K inhibitors can also improve fertility rates, since the normalization of genital structure has a positive effect on the implantation rate.

### SUMMARY OF THE INVENTION

The present invention relates to a method of treating and/or preventing endometriosis in an individual comprising administering a therapeutically effective amount of a PI3K inhibitor.

The invention further relates to a method of treating and/or preventing endometriosis by combined treatment of hormonal suppressor (e.g. GnRH antagonists, GnRH agonists, aromatase inhibitors, progesterone receptor modulators, estrogen receptor modulators) along with a PI3K inhibitor.

The invention also relates to a method of treating endometriosis-related infertility in a female comprising the administration of a therapeutically effective amount of a PI3K inhibitor, alone or in combination with other fertility drugs.

The invention finally relates to a pharmaceutical composition comprising a PI3K inhibitor, a hormonal suppressor and a pharmaceutically acceptable excipient.

### DESCRIPTION OF THE INVENTION

The following paragraphs provide definitions of the various chemical moieties that make up the compounds according to the invention and are intended to apply uniformly throughout the specification and claims unless an otherwise expressly set out definition provides a broader definition.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl(allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“Heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Preferred heterocycloalkyl include pyrrolidine, piperidine, piperazine, 1-methylpiperazine, morpholine, and the like.

“C₁-C₆-alkyl cycloalkyl” refers to C₁-C₆-alkyl groups having a cycloalkyl substituent, including cyclohexylmethyl, cyclopentylpropyl, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxy” refers to the group —C(O)OH.

“C₁-C₆-alkyl carboxy” refers to C₁-C₆-alkyl groups having an carboxy substituent, including 2-carboxyethyl and the like.

“Acyl” refers to the group —C(O)R where R includes “C₁-C₆-alkyl”, “aryl”, “heteroaryl”, “C₁-C₆-alkyl aryl” or “C₁-C₆-alkyl heteroaryl”.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to hetereoaryl groups having an acyl substituent, including 2-acetylpyridyl and the like.

“C₃-C₈-(hetero)cycloalkyl acyl” refers to 3 to 8 membered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, heterocycloalkyl “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl” or “aryl” or “hetero-aryl” or “C₁-C₆-alkyl aryl” or “C₁-C₆-alkyl heteroaryl”. Preferred alkoxy groups include by way of example, methoxy, ethoxy, phenoxy and the like.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes H, “C₁-C₆-alkyl” or “aryl” or “heteroaryl” or “C₁-C₆-alkyl aryl” or “C₁-C₆-alkyl heteroaryl”.

“C₁-C₆-alkyl alkoxycarbonyl” refers to C₁-C₅-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen or C₁-C₆-allyl or aryl or heteroaryl or “C₁-C₆-allyl aryl” or “C₁-C₆-alkyl hetero-aryl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N-methylureido)ethyl and the like.

“Carbamate” refers to the group —NRC(O)OR′ where each R, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Amino” refers to the group —NRR′ where each R, R′ is independently hydrogen or “C₁-C₆-alkyl” or “aryl” or “heteroaryl” or “C₁-C₆-allyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl amino” refers to C₁-C₅-alkyl groups having an amino substituent, including 2-(1-pyrrolidinyl)ethyl and the like.

“Ammonium” refers to a positively charged group —N⁺RR′R″, where each R,R′,R″ is independently “C₁-C₆-alkyl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₅-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-allyl heterocycloalkyl”.

“C₁-C₆-allyl sulfonyl” refers to C₁-C₅-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₅-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₅-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-allyl cycloalkyl”, “C₁-C₆-alkyl heterocycloalkyl”.

“C₁-C₆-alkyl sulfonylamino” refers to C₁-C₅-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the indi-vidual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “carbamate”, “heteroaryl”, “sulfinyl”, “sulfonyl”, “alkoxy”, “sulfanyl”, “halogen”, “carboxy”, trihalomethyl, cyano, hydroxy, mercapto, nitro, and the like. Alternatively said substitution could also comprise situations where neighbouring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

“Pharmaceutically acceptable cationic salts or complexes” is intended to define such salts as the alkali metal salts, (e.g. sodium and potassium), alkaline earth metal salts (e.g. calcium or magnesium), aluminium salts, ammonium salts and salts with organic amines such as with methylamine, dimethylamine, trimethylamine, ethylamine, triethylamine, morpholine, N-Me-D-glucamine, N,N′-bis(phenylmethyl)-1,2-ethanediamine, ethanolamine, diethanolamine, ethylenediamine, N-methylmorpholine, piperidine, benzathine (N,N′-dibenzylethylenediamine), choline, ethylene-diamine, meglumine (N-methylglucamine), benethamine (N-benzylphenethylamine), diethylamine, piperazine, thromethamine (2-amino-2-hydroxymethyl-1,3-propanediol), procaine as well as amines of formula —NR,R′,R″ wherein R, R′, R″ is independently hydrogen, alkyl or benzyl. Especially preferred salts, are sodium and potassium salts.

“Pharmaceutically acceptable salts or complexes” refers to salts or complexes of the below-identified compounds of the present invention that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. Said compounds can also be administered as pharmaceutically acceptable quaternary salts known by a person skilled in the art, which specifically include the quaternary ammonium salt of the formula —NR,R′,R″⁺ Z⁻, wherein R, R′, R″ is independently hydrogen, alkyl, or benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” refers to any compound that upon administration to the recipient, is capable of providing directly or indirectly, the activity disclosed herein.

The “tautomers” of the compounds according to Formula I are only those wherein R² and/or R⁰ are hydrogen and which display the Formulae (Ia) and (Ib).

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

“Aromatase Inhibitors” refers to drugs that inhibit the enzyme aromatase and by that lowers the level of the estradiol. Preferred aromatase inhibitors include by way of example anastrozole, letrozole, vorozole and exemestane.

“Estrogen receptor modulators (SERM)” refers to drugs that block the actions of estrogen by occupying the estrogen receptors on cells. SERMS also include estrogen receptor beta antagonists and estrogen receptor beta agonists. Preferred SERMs include by way of example Tamoxifen, Raloxifen.

“GnRH antagonists” refers to synthetic GnRH analogues, which are drugs that competitively block the pituitary GnRH receptor, which is located on the plasma membrane of gonadotrophs, inducing a rapid, reversible suppression of gonadotrophin secretion. Preferred GnRH antagonists include by way of example Cetrorelix, Ganirelix.

“GnRH agonists” refers to decapeptide modifications of the natural hormone GnRH, which are drugs that desensitize GnRH receptors of the pituitary gland at continued exposure, which causes an initial stimulation of the pituitary-ovarian axis, followed by a reduction in circulating serum gonadotrophin concentration and inhibition of ovarian function. Preferred GnRH agonists include by way of example Buserelin acetate, Nafarelin, Leuprolide, Triptolerin, Goserelin.

“PI3K inhibitor” refers to a compound, a peptide or a protein that inhibits the activity of Phosphatoinositides 3-kinases (PI3K). When the PI3K enzyme is inhibited, PI3K is unable to exert its enzymatic, biological and/or pharmacological effects. In one embodiment, the activity of PI3K alpha is inhibited. In another embodiment, the activity of PI3K beta is inhibited. In another embodiment, the activity of PI3K gamma is inhibited. In yet another embodiment, the activity of PI3K delta is inhibited. In a preferred embodiment the activity of PI3K gamma is inhibited. Such inhibitory activity can be determined by assays or animal models well-known in the art. In one embodiment, the PI3K inhibitor is a compound selected from the group consisting of Formulae (I), (II), (III), (IV), (V), (VI), (VII) and (VIII).

“Progesterone receptor modulators (SPRMs)”: The progesterone receptor, a member of the superfamily of nuclear receptors, is the receptor for progesterone that plays a pivotal role in female reproduction. Selective progesterone receptor modulators are drugs that can have agonist, antagonist or partial (mixed) agonist/antagonist activities depending upon the site of action. A preferred SPRM includes by way of example asoprisnil.

A first aspect of the present invention is to provide a method of treating and/or preventing endometriosis in an individual comprising administering a therapeutically effective amount of a PI3K inhibitor. In a preferred embodiment the individual is a human female.

In a second aspect, the invention relates to a method of treating and/or preventing endometriosis by sequential or combined treatment of hormonal suppressor (e.g. GnRH antagonists, GnRH agonists, aromatase inhibitors, progesterone receptor modulators, estrogen receptor modulators) along with a PI3K inhibitor.

Second or subsequent administrations of therapeutically effective amounts can be performed at a dosage which is the same, less than or greater than the initial or previous dose administered to the individual. Second or subsequent administrations can be administered during or prior to relapse of the endometriosis or the related symptoms. The terms “relapse” or “reoccurrence” are defined to encompass the appearance of one or more of symptoms of endometriosis.

In a third aspect, the invention relates to a method of treating endometriosis-related infertility in a female comprising the administration of a therapeutically effective amount of a PI3K inhibitor, alone or in combination with other fertility drugs

In one embodiment, the sequential or combined treatment regimen minimizes the disease by suppressing endocrine-dependent cells.

A forth aspect of the present invention consists in a pharmaceutical composition comprising a PI3K inhibitor, a hormonal suppressor (e.g. GnRH antagonists, GnRH agonists, aromatase inhibitors, progesterone receptor modulators, estrogen receptor modulators) and a pharmaceutically acceptable excipient.

A fifth aspect of the present invention consists in the use of a PI3K inhibitor in the manufacture of a medicament for the treatment and/or prevention of endometriosis.

The term “preventing”, as used herein, should be understood as partially or totally preventing, inhibiting, alleviating, or reversing one or more symptoms or cause(s) of endometriosis.

A proposed model for progression of endometriotic disease predicts that lesions progress from benign inflammatory lesions responsive to endocrine intervention to partially or completely hormonally unresponsive lesions that involve upregulated survival pathways in addition to inflammatory pathways.

Therefore, in one embodiment, the PI3K inhibitor may interfere with survival pathways in endometriosis.

A sixth aspect of the invention relates to the use of a PI3K inhibitor together with a hormonal suppressor (e.g. GnRH antagonists, GnRH agonists, aromatase inhibitors, progesterone receptor modulators, estrogen receptor modulators) and a pharmaceutically acceptable carrier in the manufacture of a medicament for the treatment and/or prevention of endometriosis.

The use of a PI3K inhibitor together with a hormonal suppressor (e.g. GnRH antagonists, GnRH agonists, aromatase inhibitors, progesterone receptor modulators, estrogen receptor modulators) can be a sequential or a combined use of the PI3K inhibitor and the hormonal suppressor.

A seventh aspect of the invention, relates to the use of a PI3K inhibitor, alone or in combination with other drugs, in the manufacture of a medicament for the treatment of endometriosis-related infertility.

An eighth aspect of the invention, relates to the use of a PI3K inhibitor for the treatment of endometriosis.

In particular, when endometriosis-related infertility is intended to be treated or cured, drugs for the treatment of infertility e.g. biologically active human chorionic gonadotrophin (hCG), luteinizing hormone (LH) or follicle stimulating hormone (FSH), either in a natural highly purified or in a recombinant form, can be administered. Such molecules and methods of their production have been described in the European Patent Applications EP 160,699, EP 211,894 and EP 322,438.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the PI3K inhibitor is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like.

Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as pepper-mint, methyl salicylate, or orange flavoring.

Injectable compositions are typically based upon injectable sterile saline or phosphate-buffered saline or other injectable carriers known in the art. As above-mentioned, the PI3K inhibitor in such compositions is typically a minor component, frequently ranging between 0.05 to 10% by weight with the remainder being the injectable carrier and the like.

The above-described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharma-ceutical Sciences.

The definition of “pharmaceutically acceptable” is meant to encompass any carrier, which does not interfere with effectiveness of the biological activity of the active ingredient and that is not toxic to the host to which it is administered. For example, for parenteral administration, PI3K inhibitor may be formulated in a unit dosage form for injection in vehicles such as saline, dextrose solution, serum albumin and Ringer's solution.

For parenteral (e.g. intravenous, subcutaneous, intramuscular) administration, PI3K inhibitors can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The therapeutically effective amounts of a PI3K inhibitor will be a function of many variables, including the type of inhibitor, the affinity of the inhibitor for PI3K, any residual cytotoxic activity exhibited by the PI3K inhibitor, the route of administration or the clinical condition of the patient.

A “therapeutically effective amount” is such that when administered, the PI3K inhibitor results in inhibition of the biological activity of PI3K. The dosage administered, as single or multiple doses, to an individual will vary depending upon a variety of factor, including PI3K inhibitor pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled, as well as in vitro and in vivo methods of determining the inhibition of PI3K in an individual.

The PI3K inhibitors may be of Formula (I)

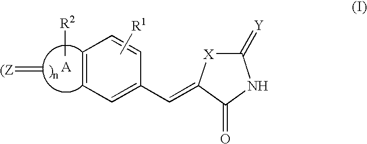

Said compounds are disclosed in WO 04/007491 (Applied Research Systems ARS Holding NV) that are described in particular for the treatment of autoimmune disorders and/or inflammatory diseases, cardiovascular diseases, neurodegenerative diseases, bacterial or viral infections, kidney diseases, platelet aggregation, cancer, transplantation complications, graft rejection or lung injuries.

In the compounds according to Formula (I) as well as its geometrical isomers, its optically active forms as enantiomers, diastereomers and its racemate forms, as well as pharmaceutically acceptable salts and pharmaceutically active derivatives thereof.

The substituents within Formula (I) are defined as follows:

A is an unsubstituted or substituted 5-8 membered heterocyclic group or an unsubstituted or substituted carbocyclic group.

Said carbocyclic group may be fused with an unsubstituted or substituted aryl, an unsubstituted or substituted heteroaryl, an unsubstituted or substituted cycloalkyl or an unsubstituted or substituted heterocycloalkyl.

Such heterocyclic or carbocyclic groups comprise aryl, heteroaryl, cycloalkyl and heterocycloalkyl, including phenyl, phenantrenyl, cyclopentyl, cyclohexyl, norbornyl, pyrrolidine, piperidine, piperazine, 1-methylpiperazine, morpholine, pyrrolyl, furanyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl,1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

Further exemplary heterocyclic or carbocyclic groups A include unsubstituted or substituted dioxol, unsubstituted or substituted dioxin, unsubstituted or substituted dihydrofuran, unsubstituted or substituted (dihydro) furanyl, unsubstituted or substituted (dihydro)oxazinyl, unsubstituted or substituted oxazinoyl, unsubstituted or substituted pyridinyl, unsubstituted or substituted isooxazolyl, unsubstituted or substituted oxazolyl unsubstituted or substituted (dihydro)napthalenyl, unsubstituted or substituted pyrimidinyl, unsubstituted or substituted triazolyl, unsubstituted or substituted imidazolyl, unsubstituted or substituted pyrazinyl, unsubstituted or substituted thiazolyl, unsubstituted or substituted thiadiazolyl, unsubstituted or substituted oxadiazolyl.

X is S, O or NH, preferably S.

Y¹ and Y² are independently from each other selected from the group consisting of S, O or —NH, preferably O.

Z is either S or O, preferably O.

R¹ is selected from the group consisting of H, CN, carboxy, acyl, C₁-C₆-alkoxy, halogen, hydroxy, acyloxy, an unsubstituted or substituted C₁-C₆-alkyl carboxy, an unsubstituted or substituted C₁-C₆-alkyl acyloxy, an unsubstituted or substituted C₁-C₆-alkyl alkoxy, alkoxycarbonyl, an unsubstituted or substituted C₁-C₆-alkyl alkoxycarbonyl, aminocarbonyl, an unsubstituted or substituted C₁-C₆-alkyl aminocarbonyl, acylamino, an unsubstituted or substituted C₁-C₆-alkyl acylamino, ureido, an unsubstituted or substituted C₁-C₆-alkyl ureido, amino, an unsubstituted or substituted C₁-C₆-alkyl amino, ammonium, sulfonyloxy, an unsubstituted or substituted C₁-C₆-alkyl sulfonyloxy, sulfonyl, an unsubstituted or substituted C₁-C₆-alkyl sulfonyl, sulfinyl, an unsubstituted or substituted C₁-C₆-alkyl sulfinyl, sulfanyl, an unsubstituted or substituted C₁-C₆-alkyl sulfanyl, sulfonylamino, an unsubstituted or substituted C₁-C₆-alkyl sulfonylamino and carbamate. In a specific embodiment R¹ is H.

R² is selected from the group consisting of H, halogen, acyl, amino, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-allyl aminocarbonyl, an unsubstituted or substituted C₁-C₆-alkyl acyloxy, an unsubstituted or substituted C₁-C₆-alkyl acylamino, an unsubstituted or substituted C₁-C₆-alkyl ureido, an unsubstituted or substituted C₁-C₆-alkyl carbamate, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, an unsubstituted or substituted aryl, an unsubstituted or substituted C₃-C₈-cycloalkyl or heterocycloalkyl, an unsubstituted or substituted C₁-C₆-alkyl aryl, an unsubstituted or substituted C₂-C₆-alkenyl-aryl, an unsubstituted or substituted C₂-C₆-alkynyl aryl, carboxy, cyano, hydroxy, C₁-C₆-alkoxy, nitro, acylamino, ureido, sulfonylamino, sulfanyl and sulfonyl.

n is an integer selected from 0, 1 or 2, preferably n is 0 or 1. Even more preferred is

n=0.

According to a specific embodiment of the invention, R¹ and R² are both H.

In a further specific embodiment according to the invention, X is S, Y¹ and Y² are both O, R¹ and R² are as above defined and n is 0.

In one embodiment the PI3K inhibitor is a thiazolidindione-vinyl fused-benzene derivatives of Formula (II):

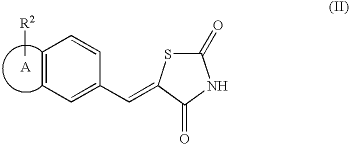

A is selected from the group consisting of unsubstituted or substituted dioxol, unsubstituted or substituted dioxin, unsubstituted or substituted dihydrofuran, unsubstituted or substituted (dihydro) furanyl, unsubstituted or substituted (dihydro)oxazinyl, unsubstituted or substituted oxazinoyl, unsubstituted or substituted pyridinyl, unsubstituted or substituted isooxazolyl, unsubstituted or substituted oxazolyl unsubstituted or substituted (dihydro)napthalenyl, unsubstituted or substituted pyrimidinyl, unsubstituted or substituted triazolyl, unsubstituted or substituted imidazolyl, unsubstituted or substituted pyrazinyl, unsubstituted or substituted thiazolyl, unsubstituted or substituted thiadiazolyl, unsubstituted or substituted oxadiazolyl.

R² is selected from the group consisting of H, halogen, acyl, amino, unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkyl carboxy, unsubstituted or substituted C₁-C₆-alkyl acyl, unsubstituted or substituted C₁-C₆-alkyl alkoxycarbonyl, unsubstituted or substituted C₁-C₆-alkyl aminocarbonyl, unsubstituted or substituted C₁-C₆-alkyl acyloxy, unsubstituted or substituted C₁-C₆-alkyl acylamino, unsubstituted or substituted C₁-C₆-alkyl ureido, unsubstituted or substituted C₁-C₆-alkyl carbamate, unsubstituted or substituted C₁-C₆-alkyl amino, unsubstituted or substituted C₁-C₆-alkyl alkoxy, unsubstituted or substituted C₁-C₆-alkyl sulfanyl, unsubstituted or substituted C₁-C₆-alkyl sulfinyl, unsubstituted or substituted C₁-C₆-alkyl sulfonyl, unsubstituted or substituted C₁-C₆-alkyl sulfonylaminoaryl, an unsubstituted or substituted aryl, unsubstituted or substituted C₃-C₈-cycloalkyl or heterocycloalkyl, unsubstituted or substituted C₁-C₆-alkyl aryl, unsubstituted or substituted C₂-C₆-alkenyl-aryl, unsubstituted or substituted C₂-C₆-alkynyl aryl, carboxy, cyano, hydroxy, C₁-C₆-alkoxy, nitro, acylamino, ureido, sulfonylamino, sulfanyl and sulfonyl.

In another embodiment, the PI3K inhibitor is a thiazolidinone-vinyl fused-benzene derivatives of Formula (II′). Compounds of Formulae (II)-(VI) as well as their synthesis are described in WO 04/007491 (Applied Research Systems ARS Holding N.V.):

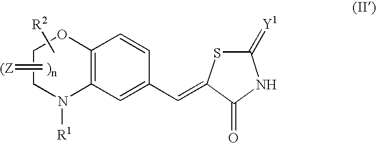

as well as its geometrical isomers, its optically active forms as enantiomers, diastereomers and its racemate forms, as well as pharmaceutically acceptable salts and pharmaceutically active derivatives thereof, wherein Y¹, Z, R¹, R² are as above defined and n is 0 or 1.

In a specific embodiment R¹ is an unsubstituted or substituted C₁-C₆-alkyl, an unsubstituted or substituted C₁-C₆-alkyl aryl, an unsubstituted or substituted aryl, an unsubstituted or substituted C₃-C₈-cycloalkyl or -heterocycloalkyl, an unsubstituted or substituted C₁-C₆-alkyl aryl, an unsubstituted or substituted C₂-C₆-alkenyl-aryl, an unsubstituted or substituted C₂-C₆-alkynyl aryl.

In another preferred embodiment, Y¹ is O.

In another embodiment, the PI3K inhibitor is a thiazolidinone-vinyl fused-benzene derivative of Formula (III):

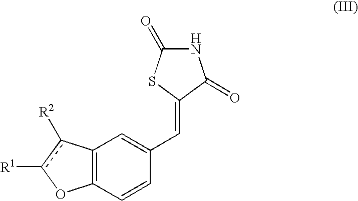

as well as its geometrical isomers, its optically active forms as enantiomers, diastereomers and its racemate forms, as well as pharmaceutically acceptable salts and pharmaceutically active derivatives thereof, wherein R¹ and R² are as above defined (the dotted line represents the optional presence of a double bond).

In another embodiment, the PI3K inhibitor is a compound of Formulae (IV), (V) and (VI):

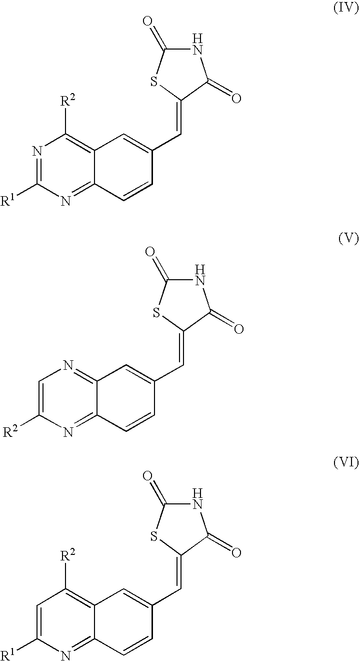

R¹ is selected from the group consisting of hydrogen, halogen, cyano, C₁-C₆-alkyl, C₁-C₆-alkoxy, acyl and alkoxy carbonyl, while R² is as above defined. In a specific embodiment R² is an amino moiety.

In another embodiment, the PI3K inhibitor is a compound of Formulae (I′), whereby R¹, Y¹ are as above defined and W is selected from O, S or —NR³ wherein R³ is H or an unsubstituted or substituted C₁-C₆ alkyl group. In a preferred embodiment, R¹ is an unsubstituted or substituted C₁-C₄ alkyl group or an unsubstituted or substituted C₁-C₅ alkenyl group, carboxy, cyano, C₁-C₄-alkoxy, nitro, acylamino, ureido.

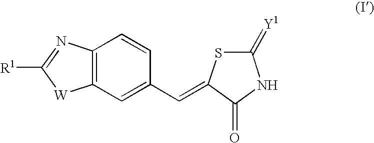

Compounds of the present invention include in particular those of the group consisting of:
5-(1,3-benzodioxol-5-ylmethylene)-1,3-thiazolidine-2,4-dione
5-(1,3-benzodioxol-5-ylmethylene)-2-thioxo-1,3-thiazolidin-4-one
5-(2,3-dihydro-1,4-benzodioxin-6-ylmethylene)-1,3-thiazolidine-2,4-dione
5-(2,3-dihydro-1-benzofuran-5-ylmethylene)-1,3-thiazolidine-2,4-dione
5-[(7-methoxy-1,3-benzodioxol-5-yl)methylene]-1,3-thiazolidine-2,4-dione
5-[(9,10-dioxo-9,10-dihydroanthracen-2-yl)methylene]-1,3-thiazolidine-2,4-dione
(5-[(2,2-difluoro-1,3-benzodioxol-5-yl)methylene]-1,3-thiazolidine-2,4-dione
(5Z)-5-(1,3-dihydro-2-benzofuran-5-ylmethylene)-1,3-thiazolidine-2,4-dione
5-(1-benzofuran-5-ylmethylene)-1,3-thiazolidine-2,4-dione
5-[(4-methyl-3-oxo-3,4-dihydro-2H-1,4-benzoxazin-6-yl)methylene]-1,3-thiazolidine-2,4-dione
5-(1,3-benzodioxol-5-ylmethylene)-2-imino-1,3-thiazolidin-4-one
5-Quinolin-6-ylmethylene-thiazolidine-2,4-dione
5-Quinolin-6-ylmethylene-2-thioxo-thiazolidin-4-one
2-Imino-5-quinolin-6-ylmethylene-thiazolidin-4-one
5-(3-Methyl-benzo[d]isoxazol-5-ylmethylene)-thiazolidine-2,4-dione
5-(4-Phenyl-quinazolin-6-ylmethylene)-thiazolidine-2,4-dione
5-(4-Dimethylamino-quinazolin-6-ylmethylene)-thiazolidine-2,4-dione
5-[(4-aminoquinazolin-6-yl)methylene]-1,3-thiazolidine-2,4-dione
5-[(4-piperidin-1-ylquinazolin-6-yl)methylene]-1,3-thiazolidine-2,4-dione
5-[(4-morpholin-4-ylquinazolin-6-yl)methylene]-1,3-thiazolidine-2,4-dione
5-{[4-(benzylamino)quinazolin-6-yl]methylene}-1,3-thiazolidine-2,4-dione
5-{[4-(diethylamino)quinazolin-6-yl]methylene}-1,3-thiazolidine-2,4-dione
5-({4-[(pyridin-2-ylmethyl)amino]quinazolin-6-yl}methylene)-1,3-thiazolidine-2,4-dione
5-({4-[(pyridin-3-ylmethyl)amino]quinazolin-6-yl}methylene)-1,3-thiazolidine-2,4-dione
ethyl 1-{6-[(2,4-dioxo-1,3-thiazolidin-5-ylidene)methyl]quinazolin-4-yl}piperidine-3-carboxylate
ethyl 1-{6-[(2,4-dioxo-1,3-thiazolidin-5-ylidene)methyl]quinazolin-4-yl}piperidine-4-carboxylate
tert-butyl 1-{6-[(2,4-dioxo-1,3-thiazolidin-5-ylidene)methyl]quinazolin-4-yl}-L-prolinate
5-{[4-(4-methylpiperazin-1-yl)quinazolin-6-yl]methylene}-1,3-thiazolidine-2,4-dione
5-{[4-(4-pyrimidin-2-ylpiperazin-1-yl)quinazolin-6-yl]methylene}-1,3-thiazolidine-2,4-dione
5-({4-[4-(4-fluorophenyl)piperidin-1-yl]quinazolin-6-yl}methylene)-1,3-thiazolidine-2,4-dione
5-{[4-(4-benzylpiperidin-1-yl)quinazolin-6-yl]methylene}-1,3-thiazolidine-2,4-dione
5-({4-[4-(2-phenylethyl)piperidin-1-yl]quinazolin-6-yl}methylene)-1,3-thiazolidine-2,4-dione
5-{[4-(4-methylpiperidin-1-yl)quinazolin-6-yl]methylene}-1,3-thiazolidine-2,4-dione
5-{[4-(4-hydroxypiperidin-1-yl)quinazolin-6-yl]methylene}-1,3-thiazolidine-2,4-dione
1-[6-(2,4-Dioxo-thiazolidin-5-ylidenemethyl)-quinazolin-4-yl]-piperidine-4-carboxylic acid
1-[6-(2,4-Dioxo-thiazolidin-5-ylidenemethyl)-quinazolin-4-yl]-piperidine-3-carboxylic acid
1-[6-(2,4-Dioxo-thiazolidin-5-ylidenemethyl)-quinazolin-4-yl]-pyrrolidine-2-carboxylic acid
5-(4-Methylamino-quinazolin-6-ylmethylene)-thiazolidine-2,4-dione
5-(4-Methoxy-quinazolin-6-ylmethylene)-thiazolidine-2,4-dione
2-Imino-5-(4-methylamino-quinazolin-6-ylmethylene)-thiazolidin-4-one
2-Imino-5-(4-piperidine-quinazolin-6-ylmethylene)-thiazolidin-4-one
2-Imino-5-(4-dimethylamino-quinazolin-6-ylmethylene)-thiazolidin-4-one
5-(2-Methyl-2H-benzotriazol-5-ylmethylene)-thiazolidine-2,4-dione
5-(3-Methyl-3H-benzotriazol-5-ylmethylene)-thiazolidine-2,4-dione
5-(3-Ethyl-3H-benzoimidazol-5-ylmethylene)-thiazolidine-2,4-dione
5-{[1-(4-phenylbutyl)-1H-benzimidazol-6-yl]methylene}-1,3-thiazolidine-2,4-dione
5-[(1-prop-2-yn-1-yl-1H-benzimidazol-6-yl)methylene]-1,3-thiazolidine-2,4-dione
5-[(1-{2-[4-(trifluoromethyl)phenyl]ethyl}-1H-benzimidazol-6-yl)methylene]-1,3-thiazolidine-2,4-dione
5-({1-[2-(4-hydroxyphenyl)ethyl]-1H-benzimidazol-6-yl}methylene)-1,3-thiazolidine-2,4-dione
methyl 4-{6-[(2,4-dioxo-1,3-thiazolidin-5-ylidene)methyl]-1H-benzimidazol-1-yl}cyclohexanecarboxylate
5-({1-[2-(5-methoxy-1H-indol-3-yl)ethyl]-1H-benzimidazol-6-yl}methylene)-1,3-thiazolidine-2,4-dione
5-({1-[(1-methyl-1H-pyrazol-4-yl)methyl]-1H-benzimidazol-6-yl}methylene)-1,3-thiazolidine-2,4-dione
5-({1-[2-(3,4-dimethoxyphenyl)ethyl]-1H-benzimidazol-6-yl}methylene)-1,3-thiazolidine-2,4-dione
5-({1-[2-(4-phenoxyphenyl)ethyl]-1H-benzimidazol-6-yl}methylene)-1,3-thiazolidine-2,4-dione
5-({1-[4-(trifluoromethyl)benzyl]-1H-benzimidazol-6-yl}methylene)-1,3-thiazolidine-2,4-dione
4-{6-[(2,4-dioxo-1,3-thiazolidin-5-ylidene)methyl]-1H-benzimidazol-1-yl}cyclohexanecarboxylic acid
5-[(1-isobutyl-1H-benzimidazol-6-yl)methylene]-1,3-thiazolidine-2,4-dione
5-({1-[2-(1,3-benzodioxol-4-yl)ethyl]-1H-benzimidazol-6-yl}methylene)-1,3-thiazolidine-2,4-dione
5-({1-[2-(2-phenoxyphenyl)ethyl]-1H-benzimidazol-6-yl}methylene)-1,3-thiazolidine-2,4-dione
5-{[1-(3,3-diphenylpropyl)-1H-benzimidazol-6-yl]methylene}-1,3-thiazolidine-2,4-dione
5-{[1-(2-methoxybenzyl)-1H-benzimidazol-6-yl]methylene}-1,3-thiazolidine-2,4-dione
5-{[1-(3-furylmethyl)-1H-benzimidazol-6-yl]methylene}-1,3-thiazolidine-2,4-dione
5-[(1-propyl-1H-benzimidazol-6-yl)methylene]-1,3-thiazolidine-2,4-dione
5-Quinoxalin-6-ylmethylene-thiazolidine-2,4-dione
5-Quinoxalin-6-ylmethylene-2-thioxo-thiazolidin-4-one
2-Imino-5-quinoxalin-6-ylmethylene-thiazolidin-4-one
5-Benzothiazol-6-ylmethylene-thiazolidine-2,4-dione
5-(3-Methyl-benzofuran-5-ylmethylene)-thiazolidine-2,4-dione
5-(2-Bromo-3-methyl-benzofuran-5-ylmethylene)-thiazolidine-2,4-dione
5-(3-bromo-benzofuran-5-ylmethylene)-thiazolidine-2,4-dione
3-[5-(2,4-Dioxo-thiazolidin-5-ylidenemethyl)-benzofuran-3-yl]-acrylic acid ethyl ester
3-[5-(2,4-Dioxo-thiazolidin-5-ylidenemethyl)-benzofuran-3-yl]-acrylic acid
5-[3-(3-Oxo-3-piperidin-1-yl-propenyl)-benzofuran-5-ylmethylene]-thiazoli-dine-2,4-dione
Methyl 1-((3-{5-[(2,4-dioxo-1,3-thiazolidin-5-ylidene)methyl]-1-benzofuran-3-yl}prop-2-enoyl)prolinate
Methyl 1-((3-{5-[(2,4-dioxo-1,3-thiazolidin-5-ylidene)methyl]-1-benzofuran-3-yl}prop-2-enoyl)-D-prolinate
(5-({3-[(3-oxo-3-pyrrolidin-1-ylprop-1-en-1-yl]-1-benzofuran-5-yl}methylene)-1,3-thiazolidine-2,4-dione
5-({3-[3-morpholin-4-yl-3-oxoprop-1-en-1-yl]-1-benzofuran-5-yl}methylene)-1,3-thiazolidine-2,4-dione
Methyl 1-(3-{5-[(2,4-dioxo-1,3-thiazolidin-5-ylidene)methyl]-1-benzofuran-3-yl}prop-2-enoyl)-L-prolinate
N-cyclohexyl-3-{5-[(2,4-dioxo-1,3-thiazolidin-5-ylidene)methyl]-1-benzofuran-3-yl}-N-methylacrylamide
3-{5-[(2,4-dioxo-1,3-thiazolidin-5-ylidene)methyl]-1-benzofuran-3-yl}-N-ethyl-N-(2-hydroxyethyl)acrylamide
N-cyclobutyl-3-{5-[(2,4-dioxo-1,3-thiazolidin-5-ylidene)methyl]-1-benzofuran-3-yl}acrylamide
5-({3-[3-azetidin-1-yl-3-oxoprop-1-en-1-yl]-1-benzofuran-5-yl}methylene)-1,3-thiazolidine-2,4-dione
5-({3-[3-(1,3-dihydro-2H-isoindol-2-yl)-3-oxoprop-1-en-1-yl]-1-benzofuran-5-yl}methylene)-1,3-thiazolidine-2,4-dione
5-({3-[3-azepan-1-yl-3-oxoprop-1-en-1-yl]-1-benzofuran-5-yl}methylene)-1,3-thiazolidine-2,4-dione
3-{5-[(2,4-dioxo-1,3-thiazolidin-5-ylidene)methyl]-1-benzofuran-3-yl}-N-piperidin-1-ylacrylamide
3-{5-[(2,4-dioxo-1,3-thiazolidin-5-ylidene)methyl]-1-benzofuran-3-yl}-N-(pyridin-3-ylmethyl)acrylamide
N-cyclohexyl-3-{5-[(2,4-dioxo-1,3-thiazolidin-5-ylidene)methyl]-1-benzofuran-3-yl}acrylamide
5-({3-[3-(4-methylpiperazin-1-yl)-3-oxoprop-1-en-1-yl]-1-benzofuran-5-yl}methylene)-1,3-thiazolidine-2,4-dione
N-cycloheptyl-3-{5-[(2,4-dioxo-1,3-thiazolidin-5-ylidene)methyl]-1-benzofuran-3-yl}acrylamide
5-({3-[3-(2,5-dihydro-1H-pyrrol-1-yl)-3-oxoprop-1-en-1-yl]-1-benzofuran-5-yl}methylene)-1,3-thiazolidine-2,4-dione
N-cyclopentyl-3-{5-[(2,4-dioxo-1,3-thiazolidin-5-ylidene)methyl]-1-benzofuran-3-yl}acrylamide
3-[5-(2,4-Dioxo-thiazolidin-5-ylidenemethyl)-benzofuran-3-yl]-propionic acid ethyl ester
3-[5-(2,4-Dioxo-thiazolidin-5-ylidenemethyl)-benzofuran-3-yl]-propionic acid
5-[3-(3-Oxo-3-piperidin-1-yl-propyl)-benzofuran-5-ylmethylene]-thiazol-idine-2,4-dione
6-(2,4-Dioxo-thiazolidin-5-ylidenemethyl)-2,3-dihydro-benzo[1,4]oxazine-4-carboxylic acid tert-butyl ester
5-(3,4-Dihydro-2H-benzo[1,4]oxazin-6-ylmethylene)-thiazolidine-2,4-dione
5-(4-Benzoyl-3,4-dihydro-2H-benzo[1,4]oxazin-6-ylmethylene)-thiazolidine-2,4-dione
5-(4-Acetyl-3,4-dihydro-2H-benzo[1,4]oxazin-6-ylmethylene)-thiazolidine-2,4-dione
6-(2,4-Dioxo-thiazolidin-5-ylidenemethyl)-benzo[1,4]oxazine-4-carboxylic acid tert-butyl ester
[6-(2,4-Dioxo-thiazolidin-5-ylidenemethyl)-3-oxo-2,3-dihydro-benzo[1,4]-oxazin-4-yl]-acetic acid methyl ester
N-Benzyl-2-[6-(2,4-dioxo-thiazolidin-5-ylidenemethyl)-3-oxo-2,3-dihydro-benzo[1,4]oxazin-4-yl]-acetamide
5-(4-Butyl-3-oxo-3,4-dihydro-2H-benzo[1,4]oxazin-6-ylmethylene)-thiazoli-dine-2,4-dione
5-(4-Benzyl-3-oxo-3,4-dihydro-2H-benzo[1,4]oxazin-6-ylmethylene)-thia-zolidine-2,4-dione
5-(2-Chloro-benzofuran-5-ylmethylene)-thiazolidine-2,4-dione
5-(3-Amino-benzo[d]isoxazol-5-ylmethylene)-thiazolidine-2,4-dione
5-(3-Phenylethynyl-benzofuran-5-ylmethylene)-thiazolidine-2,4-dione
5-Benzo[1,2,5]thiadiazol-5-ylmethylene-thiazolidine-2,4-dione
5-Benzo[1,2,5]oxadiazol-5-ylmethylene-thiazolidine-2,4-dione
5-(2-Methyl-benzofuran-6-ylmethylene)-thiazolidine-2,4-dione
5-(2-Carboxymethyl-benzofuran-6-ylmethylene)-thiazolidine-2,4-dione
5-(3-Bromo-2-fluoro-2,3-dihydro-benzofuran-6-ylmethylene)-thiazolidine-2,4-dione
5-(2-Fluoro-benzofuran-6-ylmethylene)-thiazolidine-2,4-dione.

The synthesis of compounds of Formula (I)-(VI) is described in detail in WO 04/007491.

In another embodiment the PI3K inhibitor is a compound of Formula (VII):

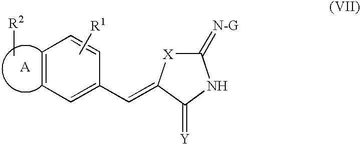

A and X are defined as above. In a preferred embodiment, A is a heterocyclic moiety.

X is either S, O or —NR³, preferably S. R³ is either H or and optionally substituted C₁-C₆-alkyl.

Y is either S or O, preferably O.

R¹ is selected from the group consisting of H, CN, carboxy, acyl, optionally substituted C₁-C₆-alkoxy, halogen, hydroxy, acyloxy, an unsubstituted or substituted C₁-C₆-alkyl carboxy, an unsubstituted or substituted C₁-C₆-alkyl acyloxy, an unsubstituted or substituted C₁-C₆-alkyl alkoxy, alkoxycarbonyl, an unsubstituted or substituted C₁-C₆-alkyl alkoxycarbonyl, aminocarbonyl, an unsubstituted or substituted C₁-C₆-alkyl aminocarbonyl, acylamino, an unsubstituted or substituted C₁-C₆-alkyl acylamino, ureido, an unsubstituted or substituted C₁-C₆-alkyl ureido, amino, an unsubstituted or substituted C₁-C₆-alkyl amino, ammonium, sulfonyloxy, an unsubstituted or substituted C₁-C₆-alkyl sulfonyloxy, sulfonyl, an unsubstituted or substituted C₁-C₆-alkyl sulfonyl, sulfinyl, an unsubstituted or substituted C₁-C₆-alkyl sulfinyl, sulfanyl, an unsubstituted or substituted C₁-C₆-alkyl sulfanyl, sulfonylamino, an unsubstituted or substituted C₁-C₆-alkyl sulfonylamino and carbamate. Preferably R¹ is H.

R² is selected from the group consisting of H, halogen, acyl, amino, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl acylamino, an unsubstituted or substituted C₁-C₆-alkyl ureido, an unsubstituted or substituted C₁-C₆-alkyl carbamate, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, an unsubstituted or substituted aryl, an unsubstituted or substituted heteroaryl, an unsubstituted or substituted C₃-C₈-cycloalkyl or heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl, carboxy, cyano, hydroxy, C₁-C₆-alkoxy, nitro, acylamino, ureido, sulfonylamino, sulfanyl and sulfonyl. Preferably R² is H. In a specific embodiment, R¹ and R² are both H.

G is selected from the group consisting of a substituted or unsubstituted C₁-C₆-alkyl, substituted or unsubstituted C₂-C₆-alkyenyl, substituted or unsubstituted C₂-C₆-alkynyl, substituted or unsubstituted heteroaryl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl, substituted or unsubstituted C₁-C₆-alkoxy, cyano, substituted or unsubstituted C₁-C₆-acyl and a sulfonyl moiety.

In a preferred embodiment, G is selected from the group consisting of a substituted or unsubstituted C₁-C₆-alkoxy, cyano and a substituted or unsubstituted sulfonyl moiety.

In another preferred embodiment, G is selected from the group consisting of a substituted or unsubstituted C₁-C₆-alkyl, including propyl and methyl; C₂-C₆-alkenyl; C₂-C₆-alkynyl and C₁-C₆-alkyl aryl, including phenyl methyl.

In another preferred embodiment, G is selected from the group consisting of an optionally substituted sulfonyl moiety, including phenyl sulfonyl, 4-methylphenyl sulfonyl, methyl sulfonyl, ethyl sulfonyl, 6-chloropyridine-3-sulfonyl, thiophene-2-carboxylic acid methyl ester-3-sulfonyl, 5-chloro-1,3-dimethyl-1H-pyrazole-4 sulfonyl, 3-chlorophenyl sulfonyl, 2-chlorophenyl sulfonyl, quinoline-8-sulfonyl, biphenyl-2-sulfonyl, pyridine-3-sulfonyl, a cyano group and an substituted or unsubstituted C₁-C₆-alkoxy.

In one embodiment G is a sulfonyl moiety of the formula —SO₂—R⁴, whereby R⁴ is selected from the group consisting of H, unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl acylamino, an unsubstituted or substituted C₁-C₆-allyl ureido, an unsubstituted or substituted C₁-C₆-alkyl carbamate, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, aryl, heteroaryl, an unsubstituted or substituted C₃-C₈-cycloalkyl or heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl -aryl or -heteroaryl, an unsubstituted or substituted C₂-C₆-alkynyl aryl or -heteroaryl, carboxy, hydroxyl, C₁-C₆-alkoxy, acylamino and sulfonylamino.

In one embodiment R⁴ is selected from the group consisting of an unsubstituted or substituted aryl, an unsubstituted or substituted heteroaryl, an unsubstituted or substituted C₁-C₃ alkyl.

In a specific embodiment, X is S; Y is O; R¹ and R² are H; and A is selected from the group consisting of a dioxolenyl, a pyridinyl or a pyrazinyl moiety, preferably a dioxolenyl and a pyridinyl moiety.

The compounds of Formula (VII) may be obtained as E/Z isomer mixture or as essentially pure E-isomers or Z isomers. The E/Z isomerism preferably refers to the vinyl moiety linking the phenyl with the azolidinone moiety. In a specific embodiment, the compounds of Formula (I) are Z-isomers.

In one embodiment, the PI3K inhibitor is selected from the group consisting of:
N-(5-Benzo[1,3]dioxol-5-ylmethylene-4-oxo-thiazolidin-2-ylidene)-2-chloro-benzenesulfonamide;
Ethanesulfonic acid (5-benzo[1,3]dioxol-5-ylmethylene-4-oxo-thiazolidin-2-ylidene)-amide;
N-(5-Benzo[1,3]dioxol-5-ylmethylene-4-oxo-thiazolidin-2-ylidene)-3-chloro-benzenesulfonamide;
5-Chloro-1,3-dimethyl-1H-pyrazole-4-sulfonic acid (5-benzo[1,3]dioxol-5-yl methylene-4-oxo-thiazolidin-2-ylidene)-amide;
3-(5-Benzo[1,3]dioxol-5-ylmethylene-4-oxo-thiazolidin-2-ylidenesulfamoyl)-thiophene-2-carboxylic acid methyl ester;
6-Chloro-pyridine-3-sulfonic acid (5-benzo[1,3]dioxol-5-ylmethylene-4-oxo-thiazolidin-2-ylidene)-amide;
Quinoline-8-sulfonic acid (5-benzo[1,3]dioxol-5-ylmethylene-4-oxo-thiazolidin-2-ylidene)-amide;
N-(5-Benzo[1,3]dioxol-5-ylmethylene-4-oxo-thiazolidin-2-ylidene)-benzene sulfonamide;
N-(5-Benzo[1,3]dioxol-5-ylmethylene-4-oxo-thiazolidin-2-ylidene)-4-methyl-benzenesulfonamide;
N-(5-Benzo[1,3]dioxol-5-ylmethylene-4-oxo-thiazolidin-2-ylidene)-methane sulfonamide;
N-[5-(2,2-Difluoro-benzo[1,3]dioxol-5-ylmethylene)-4-oxo-thiazolidin-2-ylidene]-benzenesulfonamide;
N-[5-(2,2-Difluoro-benzo[1,3]dioxol-5-ylmethylene)-4-oxo-thiazolidin-2-ylidene]-4-methyl-benzenesulfonamide;
N-[5-(2,2-Difluoro-benzo[1,3]dioxol-5-ylmethylene)-4-oxo-thiazolidin-2-ylidene]-methanesulfonamide;
Biphenyl-2-sulfonic acid (5-benzo[1,3]dioxol-5-ylmethylene-4-oxo-thiazolidin-2-ylidene)-amide

Pyridine-3-sulfonic acid (5-benzo[1,3]dioxol-5-ylmethylene-4-oxo-thiazolidin-2-ylidene)-amide;
3-(4-Oxo-5-quinolin-6-ylmethylene-thiazolidin-2-ylidenesulfamoyl)-thiophene-2-carboxylic acid methyl ester;
2-Chloro-N-(4-oxo-5-quinolin-6-ylmethylene-thiazolidin-2-ylidene)-benzene sulfonamide;
3-(5-Benzo[1,3]dioxol-5-ylmethylene-4-oxo-thiazolidin-2-ylidenesulamoyl)-thiophene-2-carboxylic acid;
5-Benzo[1,3]dioxol-5-ylmethylene-4-oxo-thiazolidin-2-ylidene-cyanamide;
5-Benzo[1,3]dioxol-5-ylmethylene-thiazolidine-2,4-dione 2-(O-methyl-oxime);
4-Oxo-5-quinoxalin-6-ylmethylene-thiazolidin-2-ylidene-cyanamide;
5-Benzo[1,3]dioxol-5-ylmethylene-2-benzylimino-thiazolidin-4-one;
2-Benzylimino-5-quinolin-6-ylmethylene-thiazolidin-4-one;
2-Propylimino-5-quinolin-6-ylmethylene-thiazolidin-4-one;
5-Benzo[1,3]dioxol-5-ylmethylene-2-propylimino-thiazolidin-4-one;
5-(4-Dimethylamino-quinazolin-6-ylmethylene)-2-methylamino-thiazol-4-one.

The synthesis of compounds of Formula (VII) is described in detail in WO 05/011686 (Applied Research Systems, ARS Holding N.V.).

In another embodiment the PI3K inhibitor may be a compound as shown in Formula (VIII) (WO 04/17950, Piramed):

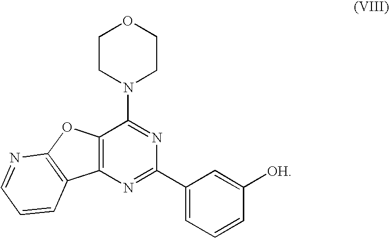

The present invention will now be illustrated by the example, which is not intended to be limiting in any way.

### EXAMPLES

### Example 1

### Models for Endometriosis

The effect of PI3K inhibitors was evaluated in both in vitro and in vivo models of endometriosis. The efficacy of the drug treatment in inhibiting endometriosis was tested in two in vivo models, i) nude mouse model and ii) the rat model.

### Example 1.1

### Induction of Cell-Death in Endometriotic Cells

It is well established that the glandular and stromal tissues from the eutopic endometrium implant in ectopic sites leading to endometriosis. Survival of the ectopic implants is due to a reduced cell death (apoptosis) of these implants, and is presumed to be due to increased expression of survival cell signaling pathways. Proteins or specific small molecule compounds that induce target-specific cell-death of ectopic endometriotic cells without affecting eutopic endometrium or other normal cells could be used as a treatment for eliminating endometriosis. In this regard, we examined the effect of PI3K inhibitor-1 (5-Quinoxalin-6-ylmethylene-thiazolidine-2,4-dione) on its ability to induce cell death of endometriotic cells (12Z cells), an immortalized human epithelial endometriotic cell (Zeitvogel et al. 2001). These cells grow in culture and secrete cytokines in response to TNFα that have been reported to be elevated in the peritoneal fluid of endometriotic patients. The 12Z cells were treated with PI3K inhibitor-1 for 24 h and cell death was measured by crystal violet staining. Since the peritoneal fluids contain elevated concentrations of TNFα, the inhibitor was also tested in the presence of this cytokine. Dead cells get detached from the tissue culture plate and washed away during staining with crystal violet. The intensity of the color will determine the extent of live cells present in the plate. PI3K inhibitor-1 was tested in these cells and was found to reduce the amount of live cells in culture, either alone or in the presence of TNFα. EC50 of the inhibitor is provided in Table 1. The extent of cell death induced by the inhibitor was greater in the presence of TNFα. TNFα alone had no effect of cell viability (data not shown).

TABLE 1
Concentration of PI3K inhibitor-1 required to induce 50% cell
death (EC50 in μM) of human endometriotic cells. Cells were
incubated with varying concentrations of inhibitor-1 alone or
in the presence of 15 ng/ml of TNFα for 24 h and cell viability
was measured using crystal violet staining.

| | Alone [μM] | +TNFα [μM] |
|---|---|---|
| Inhibitor-1 | >10 | 6.40 |

### Example 1.2

### Nude Mouse Model

Human endometrial tissue was injected in ovarectomized nude mice to establish the disease (Bruner-Tran et al. 2002). In brief, endometrial biopsies obtained from normal volunteers or from endometriotic patients were cut into small pieces and cultured in the presence of estradiol for 24 h. Treated tissues, were injected either subcutaneously or intraperitoneally into ovarectomized nude mice with estradiol implant. Within 2-4 days of injection, ectopic endometriotic lesions develop in animals. Treatment with either progesterone or PI3K inhibitor-1 was started 10 days following the injection of tissue. The compound was administered at a dose of 10 mg/kg and 30 mg/kg/animal for 28 days. Earlier work using this model has established that progesterone treatment prevents disease progression, hence this was used as control. Following the completion of treatment, animals were sacrificed, lesions developed from the transplanted tissue found in both subcutaneous and intraperitoneal sites, were measured (both size and number).

Table 2 below, illustrates the results of studies carried out in nude mice. The PI3K inhibitor at a dose of 10 mg/kg and 30 mg/kg was effective in regressing the established disease by 53% and 80% respectively compared to progesterone treatment. The mean lesion size was also reduced by 10% and 30% respectively by the treatment. These results are significant, since the model measures the growth/regression of human endometrial tissue and thus has a direct relevance for treating the human disease. PI3K inhibitor treatment had no effect on the uterine weight and size of the animals.

TABLE 2
Effects of PI3K inhibitor-1 on the regression of endometriotic
lesions in the xenograft nude mouse model.

| | Lesion (% Progesterone) | |
|---|---|---|
| | Compared to progesterone | |
| Treatment | treated group | Lesion size |
| PI3K inhibitor-1 | 53% Decrease | 10% Decrease |
| 10 mg/kg × 28 days | | |
| PI3K inhibitor-1 | 80% Decrease | 30% Decrease |
| 30 mg/kg × 28 days | | |

### Example 1.3

### Rat Model

Endometriosis was induced in rats as described earlier (D'Antonio et al 2000). In brief, autologous uterine horn fragment was transplanted onto the inner surface of the abdominal wall in rat. Three weeks following transplantation, the size and the viability of the engrafted tissue was measured. One week after the confirmation of the tissue attachment, treatments were started. The control group received the vehicle, the PI3K inhibitor-1 was administered orally (po) at a single dose of 30 mg/kg per day. Treatment with the inhibitor was conducted for nine days, animals were anaesthetized 2 hr following the last treatment and blood samples were collected. Surface area of the endometriosis-like foci was measured. The endometriotic-like foci was removed for histology.

Inhibitor-1 significantly induced regression (64%) of established endometriotic lesions. Treatment with antide (for comparison) caused 94% regression (data not shown). These results from in vitro and in vivo models suggest an important activity of the molecule directly on the endometriotic tissue. Another important distinction of the rat model is that intact myometrial and endometrial tissues are surgically resected into experimental animals.

Taken together results from in vitro studies and these two in vivo model systems show that kinase inhibitors, which target PI3K pathway are effective agents for treatment of endometriosis.

### REFERENCE LIST

Bruner-Tran et al. (2002) Ann NY Acad. Sci., 955:328-339
Cantley, 2000, Science, 296, 1655-1657
D'Antonio et al. (2000) J. Reprod Immunol. 48:81-98
Dawood, M. Y et al. (1993) Int. J. Gynaecol. Obstet. 40 (Suppl.), 29-42
Giudice et al. (2004) Lancet 364, 1789-99
Kyama et al. (2003) Reprod Biol Endocrinol. 1, 123
Vanhaesebroeck et al., 2001, Annu. Rev. Biochem., 70, 535-602
Waller et al. (1993) Fertil. Steril. 59, 511-515
Zeitvogel et al. (2001) Am. J. Pathol. 159 1839-52
EP 160,699
EP 211,894
EP 322,438
WO 04/17950
WO 04/007491
WO 05/011686

1. A method of treating and/or preventing endometriosis in an individual comprising administering a therapeutically effective amount of a PI3K inhibitor.

2. The method according to claim 1, wherein said PI3K inhibitor is administered in combination with a hormonal suppressor.

3. The method according to claim 1, wherein said hormonal suppressor is selected from the group consisting of a GnRH antagonist, GnRH agonist, aromatase inhibitor, progesterone receptor modulator and an estrogen receptor modulator.

4. The method according to claim 1, wherein said PI3K inhibitor is administered alone or in combination with drugs for the treatment of endometriosis-related infertility.

5. The method according claim 1, wherein said PI3K inhibitor is a compound according to Formula (I): 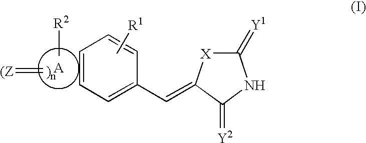
as well as its geometrical isomers, its optically active forms as enantiomers, diastereo-mers and its racemate forms, as well as pharmaceutically acceptable salts and pharma-ceutically active derivatives thereof, wherein
A is a 5-8 membered heterocyclic or carbocyclic group, wherein said carbocyclic group may be fused with aryl, heteroaryl, cycloalkyl or heterocycloalkyl;
X is S, O or NH;
Y¹ and Y² are each independently selected from the group consisting of S, O or —NH;
Z is either S or O; and
R¹ is selected from the group consisting of H, CN, carboxy, acyl, C₁-C₆-alkoxy, halogen, hydroxy, acyloxy, C₁-C₆-alkyl carboxy, C₁-C₆-alkyl acyloxy, C₁-C₆-alkyl alkoxy, alkoxycarbonyl, C₁-C₆-alkyl alkoxycarbonyl, aminocarbonyl, C₁-C₆-alkyl aminocarbonyl, acylamino, C₁-C₆-alkyl acylamino, ureido, C₁-C₆-alkyl ureido, amino, C₁-C₆-alkyl amino, ammonium, sulfonyloxy, C₁-C₆-alkyl sulfonyloxy, sulfonyl, C₁-C₆-alkyl sulfonyl, sulfinyl, C₁-C₆-alkyl sulfinyl, sulfanyl, C₁-C₆-alkyl sulfanyl, sulfonylamino, C₁-C₆-alkyl sulfonylamino and carbamate;
R² is selected from the group consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, C₃-C₈-cycloalkyl or heterocycloalkyl, C₁-C₆-alkyl aryl, C₂-C₆-alkenyl-aryl, C₂-C₆-alkynyl aryl, carboxy, cyano, hydroxy, C₁-C₆-alkoxy, nitro, acylamino, ureido, C₁-C₆-alkyl carbamate, sulfonylamino, sulfanyl and sulfonyl;
n is 0, 1 or 2;

6. The method according to claim 5, wherein Y¹ and Y² are both O.

7. The method according to claim 5, wherein n is either 1 or 2; and R¹ and R² are both H.

8. The method according to claim 5, wherein X is S; Y¹ and Y² are both O; R¹ and R² are as above-defined and n is 0.

9. The method according to claim 1, wherein said PI3K inhibitor is a compound according to Formula (I′): 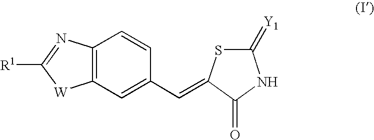
as well as its geometrical isomers, its optically active forms as enantiomers, diastereo-mers and its racemate forms, as well as pharmaceutically acceptable salts and pharmaceutically active derivatives thereof, wherein R¹, Y¹ are as defined in above, and W is selected from O, S, —NR³ wherein R³ is H or an unsubstituted or substituted C₁-C₆ alkyl group.

10. The method according to claim 1, wherein said PI3K inhibitor is a compound according to Formula (II): 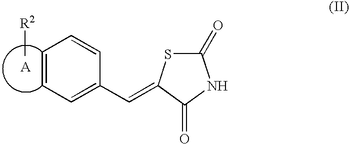
as well as its geometrical isomers, its optically active forms as enantiomers, diastereo-mers and its racemate forms, as well as pharmaceutically acceptable salts and pharma-ceutically active derivatives thereof, wherein
A is selected from the group consisting of dioxol, dioxin, dihydrofuran, (dihydro) furanyl, (dihydro)oxazinyl, pyridinyl, isooxazolyl, oxazolyl (dihydro)napthalenyl, pyrimidinyl, triazolyl, imidazolyl, pyrazinyl, thiazolidinyl, thiadiazolyl and oxadiazolyl;
R² is selected from the group consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl carbamate, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, C₃-C₈-cycloalkyl or heterocycloalkyl, C₁-C₆-alkyl aryl, C₂-C₆-alkenyl-aryl, C₂-C₆-alkynyl aryl, carboxy, cyano, hydroxy, C₁-C₆-alkoxy, nitro, acylamino, ureido, sulfonylamino, sulfanyl and sulfonyl.

11. The method according to claim 1, wherein said PI3K inhibitor is a compound according to Formula (II′): 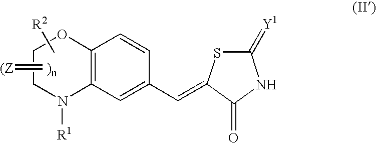
as well as its geometrical isomers, its optically active forms as enantiomers, diastereo-mers and its racemate forms, as well as pharmaceutically acceptable salts and pharma-ceutically active derivatives thereof, wherein:
Z, Y¹, R¹, R² are as above defined, n is 0 or 1.

12. The method according to claim 11, wherein Y¹ is O.

13. The method according to claim 11, wherein R¹ is selected in the group consisting of C₁-C₆-alkyl, C₁-C₆-alkyl aryl, aryl, C₃-C₈-cycloalkyl, heterocycloalkyl, C₁-C₆-alkyl aryl, C₂-C₆-alkenyl aryl and C₂-C₆-alkynyl aryl.

14. The method according to claim 1, wherein said PI3K inhibitor is a compound according to Formula (III): 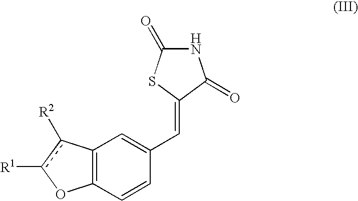
as well as its geometrical isomers, its optically active forms as enantiomers, diastereo-mers and its racemate forms, as well as pharmaceutically acceptable salts and pharmaceutically active derivatives thereof, wherein
R¹ and R² are as above defined.

15. The method according to claim 1, wherein said PI3K inhibitor is a compound according to any one of Formulae (IV), (V) and (VI):

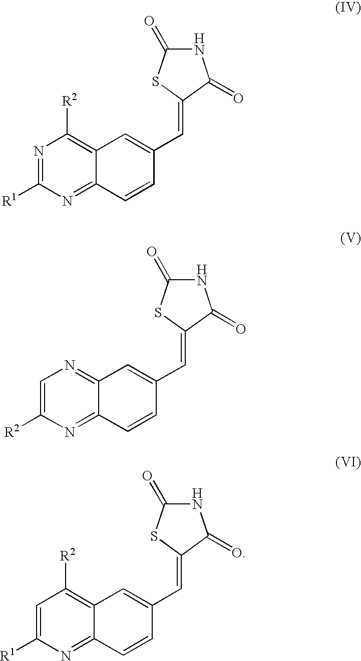

16. The method according to claim 15, wherein said PI3K inhibitor is 5-Quinoxalin-6-ylmethylene-thiazolidine-2,4-dione.

17. The method according to claim 1, wherein said PI3K inhibitor is a compound according to Formula (VII): 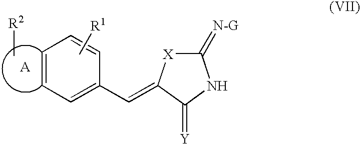
as well as its geometrical isomers, its optically active forms as enantiomers, diastereo-mers and its racemate forms, as well as pharmaceutically acceptable salts and pharmaceutically active derivatives thereof, wherein
A is an 5-8 membered heterocyclic group or an carbocyclic group which may be fused with an aryl, an heteroaryl, an cycloalkyl or an heterocycloalkyl;
X is S, O or —NR³;
Y is either S or O;
R¹ is selected from the group consisting of H, CN, carboxy, acyl, C₁-C₆-alkoxy, halogen, hydroxy, acyloxy, C₁-C₆-alkyl carboxy, C₁-C₆-alkyl acyloxy, C₁-C₆-alkyl alkoxy, alkoxycarbonyl, C₁-C₆-alkyl alkoxycarbonyl, aminocarbonyl, C₁-C₆-alkyl aminocarbonyl, acylamino, C₁-C₆-alkyl acylamino, ureido, C₁-C₆-alkyl ureido, amino, C₁-C₆-alkyl amino, ammonium, sulfonyloxy, C₁-C₆-alkyl sulfonyloxy, sulfonyl, C₁-C₆-alkyl sulfonyl, sulfinyl, C₁-C₆-alkyl sulfinyl, sulfanyl, C₁-C₆-alkyl sulfanyl, sulfonylamino, C₁-C₆-alkyl sulfonylamino and carbamate;
R² is selected from the group consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl carbamate, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl, C₂-C₆-alkynyl aryl or -heteroaryl, carboxy, cyano, hydroxy, C₁-C₆-alkoxy, nitro, acylamino, ureido, sulfonylamino, sulfanyl and sulfonyl;
G is selected from the group consisting of C₁-C₆-alkoxy, C₁-C₆-alkyl, C₂-C₆-alkenyl, C₂-C₆-alkynyl, C₁-C₆-alkyl aryl, cyano and a sulfonyl moiety; and
R³ is either H or C₁-C₆-alkyl.

18. The method according claim 17, wherein A is selected from the group consisting of 2H-(benzo-1,3-dioxolanyl), 2H, 3H-benzo-1,4-dioxanyl, 2,3-dihydrobenzofuranyl, anthraquinonyl, 2,2-difluorobenzo-1,3-dioxolenyl, 1,3-dihydrobenzofuranyl, benzofuranyl, 4-methyl-2H-benzo-1,4-oxazin-3-onyl, pyridinyl, pyrazinyl, 4-methyl-2H and 3H-benzo-1,4-oxazinyl.

19. The method according to claim 17, wherein A is either a dioxolenyl or a pyridinyl moiety.

20. The method according to claim 17, wherein R¹ and/or R² are H.

21. The method according to claim 17, wherein G is selected from the group consisting of C₁-C₆-alkoxy, cyano or a sulfonyl moiety.

22. The method according to claim 17, G is a sulfonyl moiety of the formula —SO₂—R⁴, whereby R⁴ is selected from the group consisting of H, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl carbamate, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl, C₂-C₆-alkynyl aryl or -heteroaryl, carboxy, hydroxy, C₁-C₆-alkoxy, acylamino and sulfonylamino.

23. The method according to claim 22, wherein R⁴ is selected from the group consisting of aryl, heteroaryl and C₁-C₆ alkyl.

24. The method according to claim 17, wherein X is S; Y is O; R¹ and R² are H, and A is either a dioxolenyl or pyridinyl moiety.

25. A pharmaceutical composition comprising the PI3K inhibitor of the Formula (I) of claim 5, a hormonal suppressor and a pharmaceutically acceptable excipient.

26. The pharmaceutical composition according to claim 25, wherein said hormonal suppressor is selected from the group consisting of a GnRH antagonist, GnRH agonist, aromatase inhibitor, progesterone receptor modulator and an estrogen receptor modulator.

27. (canceled)

28. The pharmaceutical composition according to claim 25, wherein said PI3K inhibitor is 5-Quinoxalin-6-ylmethylene-thiazolidine-2,4-dione.
